(19) United States
(12) Patent Application Publication
(10) Pub. No.: US 2025/0295397 A1
(43) Pub. Date: Sep. 25, 2025

(54) INTERFACE FOR CONNECTING A DRIVE UNIT TO A MEDICAL INSTRUMENT

(71) Applicant: KARL STORZ SE & Co. KG, Tuttlingen (DE)

(72) Inventors: Martin HOFFMANN, Berlin (DE); Torben KLINGELS, Landshut (DE)

(73) Assignee: KARL STORZ SE & Co. KG, Tuttlingen (DE)

(21) Appl. No.: 19/082,621

(22) Filed: Mar. 18, 2025

(30) Foreign Application Priority Data
Mar. 19, 2024 (DE) 10 2024 107 841.3

(51) Int. Cl. A61B 17/00; A61B 34/30; A61B 46/10

(52) U.S. Cl. CPC A61B 17/00; A61B 34/30; A61B 46/10; A61B 2017/00477

(57) ABSTRACT

The disclosure relates to an interface for connecting a drive unit to a medical instrument. The interface comprises at least one drive-side coupling device with at least one drive-side coupling surface, and at least one instrument-side coupling device, complementary to the drive-side coupling device, with at least one instrument-side coupling surface. The drive-side and instrument-side coupling devices can be mechanically coupled to one another with the interposition of a continuous medical barrier. At least the drive-side coupling device comprises at least one drive-side piston assembly with at least one piston which is adapted to be moved alternately in opposite directions.

### CROSS-REFERENCE TO RELATED APPLICATIONS

This application claims priority of German Patent Application No. DE 10 2024 107 841.3 filed on Mar. 19, 2024, the contents of which are incorporated herein.

### TECHNICAL FIELD

The present disclosure relates to an interface for connecting a drive unit to a medical instrument, in particular as part of a medical robot system.

### BACKGROUND

Medical robot systems, in particular, in the form of conventional medical master-slave robot systems, which are used, for example, for the mechanical guidance of medical and/or surgical instruments and the control of end effectors thereof, usually have a releasable connection at an interface between motor drives on the robot side and the surgical instruments. In this case, the drive unit and the surgical instrument are usually coupled to each other in a force-fitting manner via a gear arrangement.

In order to meet the requirements for a sterile treatment environment, a medical barrier comprising a plastic film is provided in the region of the interface, which plastic film separates a drive-side part of the interface, which usually is not sterile, from an instrument-side sterile part of the interface. For the sterile coupling of an interface or for the transmission of at least one driving force and/or at least one driving torque between the components of the interface, known medical barriers have complexly shaped films or specifically embedded adapters in the film via which the driving force and/or the driving torque can be transmitted. Such interfaces or medical barriers developed for them are described, for example, in the documents WO 2007/126443 A2 and US 2023/0390016 A1. The complexly shaped films and/or adapters integrated into the film increase the cost of the medical barrier. Furthermore, the barriers must be placed precisely, which increases the amount of work required prior to a medical and/or surgical procedure.

### SUMMARY

Based upon the prior art, it is the object of the disclosure, in particular but not limited thereto, to advantageously further develop an interface for connecting a drive unit to a medical instrument, in particular with regard to costs and/or installation or deinstallation. Furthermore, it is, in particular, an object of the present disclosure to provide a reliable and functional interface with optimized properties so that a sterile working field can always be guaranteed or ensured, in particular, before a medical procedure and/or during a medical procedure on a patient.

This object is achieved according to the disclosure by the features of the independent claims. Further developments of the disclosure can be found in the dependent claims.

The disclosure relates to an interface for connecting a drive unit to a medical instrument, comprising:
at least one drive-side coupling arrangement with at least one drive-side coupling surface, and
at least one instrument-side coupling device, complementary to the drive-side coupling at least one instrument-side coupling surface, wherein the drive-side and instrument-side coupling devices can be mechanically coupled to one another with the interposition of a continuous medical barrier, and wherein at least the drive-side coupling device comprises at least one piston assembly with at least one piston, which is adapted to be moved alternately in opposite directions.

Such a design makes it possible to provide an advantageously further developed interface. In particular, installation and deinstallation of medical instruments can be simplified, and costs and downtimes can be reduced, since a continuous medical barrier can be used, which, in particular, is simply shaped and/or can omit specially embedded adapters for transmitting a driving force and/or a driving torque. This makes it possible to eliminate the need for time-consuming, precise placement of the medical barrier. In addition, the cost of each medical application can be reduced because a single-use medical barrier can be kept as cost-effective as possible. In particular, a standard medical barrier can be used. Furthermore, the features according to the disclosure can provide a particularly reliable and functional interface with optimized properties, so that a sterile working field is always guaranteed and a reliable mechanical coupling is ensured, in particular before a medical procedure and/or during a medical procedure on a patient.

The interface may be part of a medical robot system. The robot system may have at least one robot arm, an operating console, an electronics cabinet, in particular an electronics rack, e.g., for accommodating additional devices such as an RF generator, a display unit, a patient bed, a storage unit for various medical instruments for use with the interface, and/or other units that appear useful to the person skilled in the art. The continuous medical barrier may also be part of the medical robot system. In an operating state, the interface can be carried by the robot arm and be movable thereby.

The medical instrument may be any effector that appears appropriate to a person skilled in the art, in particular, in the form of a medical and/or surgical instrument, which can preferably be carried by the robot arm and be movable thereby. The medical instrument may be permanently or releasably connected to the instrument-side coupling device or may be part of the instrument-side coupling device. The medical instrument may, for example, have a laparoscopic unit, an endoscope, a microscope, an exoscope, a scalpel, a drill, a scraper, a clamp, a pair of forceps, a pair of scissors, a syringe, a catheter, and/or other units that appear appropriate to a person skilled in the art, which can be driven and/or actuated by means of the drive unit via the interface.

In the coupled state of the interface, the drive-side coupling surface contacts a first side of the medical barrier, and the instrument-side coupling surface contacts a second side of the medical barrier opposite the first side. In this case, the coupling surfaces can preferably be designed to be complementary to one another, in particular to enable a positive reception and/or a positive clamping of the medical barrier between the connecting elements. The coupling surfaces can each have a total surface area of at least 50 mm², in particular of at least 100 mm² and for example of at least 250 mm².

A “coupling device” may in particular be a mechanical apparatus adapted to connect two or more component parts to one another and/or to be coupled to one or more component parts. The coupling device may be adapted to establish a fixed connection between the component parts so that they can interact with each other, be it mechanically, electrically, hydraulically, and/or in another way. The connecting devices, when assembled with their coupling surfaces, can form a body that is at least partially rotationally symmetrical about an axis of rotational symmetry, in particular at least partially circular-cylindrical and/or at least partially frustoconical. The coupling devices can be assembled at least partially transversely to the axis of rotational symmetry. In some embodiments, the drive-side coupling device may be larger than the instrument-side coupling device. The drive-side coupling device may have a receiving space for receiving the instrument-side coupling device.

The term “adapted” shall be understood to mean specifically programmed, provided, configured, designed, and/or equipped. The fact that an object is provided for a specific function shall further be understood, in particular, to mean that the object fulfills and/or executes this specific function in at least one application and/or operating state.

The continuous medical barrier is free of openings, inserts, and/or special embedded transmission elements for transmitting a driving force and/or a driving torque. The continuous medical barrier may have a thickness of at most 1 mm over its entire surface area, in particular of at most 0.5 mm and for example of at most 0.1 mm. The continuous medical barrier is preferably designed to be flexible. Particularly preferably, the continuous medical barrier is designed as a correspondingly processed and/or coated fabric and/or preferably as a film, in particular as a plastic film.

The fact that the drive-side and instrument-side coupling devices can be mechanically coupled to one another shall be understood in the context of the present disclosure to mean, in particular, that the drive-side and instrument-side coupling devices are adapted to be connected to one another in a captive and/or functional manner in order to enable a specific function. Through the mechanical coupling or connection, forces and/or movements can be transmitted between the two coupling devices directionally or bi-directionally, which enables the two coupling devices to work together or influence each other.

The drive-side coupling device and/or the instrument-side coupling device may comprise a housing. The housing may, in particular, be designed to protect components that are at least partially surrounded by the housing against external influences such as dust, moisture, and/or mechanical damage. The housing may be made of various materials such as plastic, metal, or rubber. The housing may be configured so that it can be easily connected to at least one other housing or system in a captive manner.

According to the disclosure, the drive-side coupling device comprises at least one piston assembly. The at least one piston assembly may, in particular, be arranged in a drive-side housing. A “piston assembly” is to be understood, in particular, as the positioning and/or arrangement of pistons in the at least one drive-side coupling device. A piston may, in particular, be a component, preferably a cylindrical component, which is at least partially or completely movable along a reference axis. In some embodiments, the at least one piston may also be designed as a fluid and/or gas column that is adapted to be deformed hydraulically and/or pneumatically. Preferably, the reference axis on which the piston is movable coincides with its longitudinal axis. The piston may be part of a hydraulic, pneumatic, or other advantageous mechanical system. In particular, the at least one piston is designed to be moved alternately in opposite directions. Preferably, the at least one piston is designed to be moved back and forth, in particular up and down, along the reference axis in a piston movement. In particular, the piston movement can be aperiodic.

The movement of the at least one piston can take place, in particular, along a reference axis which runs obliquely or perpendicular to the drive-side coupling surface. In other words, the reference axis along which the at least one piston may be movable may not run parallel to the drive-side coupling surface.

According to a further development, the piston assembly may comprise at least one further piston which is interdependently coupled to the at least one piston. “Interdependently coupled” shall mean, in particular, that the at least one piston can be coupled to the at least one further piston such that the pistons influence each other, in particular with regard to their position. The position and/or behavior or functioning of each piston depends upon the other coupled piston(s) within a coupling assembly. With regard to the movement behavior of two interdependently coupled pistons, this means that their movements are opposite to each other. In other words, the at least one piston moves in a first direction when the further piston interdependently coupled to the at least one piston moves in a second direction which is opposite to the first direction, and vice versa. The movement of at least one piston can control a movement of the instrument, and the movement of at least one further piston can correspondingly control the opposite movement of the instrument.

In some embodiments, the interface of the type described above may comprise at least one lever device. In this case, the at least one piston can be actuated together with the at least one further piston via the at least one lever device. The lever device may, in particular, be a rocker arm which is designed to bring about a linear movement of the piston or pistons. The lever device may comprise at least a first lever end and at least a second lever end. The lever ends may be rotatable around a fulcrum. A piston may be arranged on each of these lever ends. By displacing the lever device or the lever ends around the fulcrum, pistons arranged on the lever ends can be displaced. In particular, the lever device may be configured such that the lever ends extend in opposite spatial directions. The at least one piston and/or the at least one further piston may comprise an underside on which it is coupled to the lever device such that its orientation in relation to the lever device can be varied, in particular, during a movement or tilting of the lever device. In some embodiments, the at least one piston and/or the at least one further piston can be articulately coupled to the at least one lever device or its ends. The at least one piston and/or the at least one further piston can contact the respective ends of the lever device.

According to a further embodiment, the at least one piston assembly may comprise at least one cylinder guide which is designed to guide the at least one piston and/or the at least one further piston during a movement of the piston along a guide axis. A cylinder guide may, in particular, be a mechanical component that is designed to guide and/or stabilize the movement of a piston along an advantageous or defined guide path. In particular, the cylinder guide may be adapted to allow an axial movement of a piston guided therein, and at the same time to prevent and/or limit a lateral movement. The cylinder guide may be designed as a hollow cylinder. The shape of the cylinder guide can vary depending upon the shape or contour of the piston to be guided. In some embodiments, the at least one cylinder guide may be designed in the form of at least one material recess or through-bore in a monolithic structural element.

In a further embodiment, the at least one cylinder guide may have at least one edge which is at least partially and preferably completely rounded. Furthermore, the at least one piston and/or the at least one further piston may have at least one end face, the edge of which is rounded at least in sections, preferably completely. End surfaces are to be understood herein as the surfaces which, in the coupled state, are directed towards the interpositioned medical barrier.

Rounded edges have the particular advantage of preventing or at least minimizing damage and/or wear within the interface. In particular, damage to the medical barrier and potentially associated contamination can be advantageously avoided.

According to a further development, the piston assembly comprises at least one spring device which is adapted to axially preload the at least one piston and/or the at least one further piston in a spring-loaded position, in order to transmit a reliable transmission of movement to a medical instrument. The spring device may be arranged between a piston and the lever device. In some embodiments, the spring device may also be arranged within the at least one piston and/or the at least one further piston. The spring device may, in particular, be adapted to generate a preload force which presses the piston to be preloaded into a certain position and/or against another component, in particular a complementary piston. This allows constant contact between two interacting components, in particular two interacting pistons, to be achieved and reliable operation to be ensured. The spring device may, in particular, comprise a metallic and/or elastomeric coil spring, a hydraulic spring, and/or a pneumatic spring.

In some embodiments, the interface of the type described above may comprise a lifting device which is adapted to operatively couple at least the drive-side piston device and/or the drive-side coupling device to the instrument-side coupling device. A functional coupling can be achieved by displacing the drive-side coupling device and/or the instrument-side coupling device by means of one or more lifting devices, preferably in an automated manner, in the direction of the respective other coupling device, and/or bringing the coupling device into engagement with it. A lifting device is to be understood here, in particular, as a mechanical, hydraulic, and/or pneumatic apparatus which enables a preferably automated coupling or decoupling of the drive-side coupling device with the instrument-side coupling device. By means of such a lifting device, precise movements can be carried out. According to some embodiments, the lifting device may be adapted to displace at least one piston assembly relative to the drive-side housing and/or to reversibly lock it in a predetermined position. In this way, not only can the coupling or decoupling of the interface be simplified, but errors or damage due to incorrect assembly can also be prevented, or at least such a risk reduced.

According to a further development, the drive-side coupling device may comprise a plurality of piston assemblies. Each piston assembly may be assigned a degree of freedom of the medical instrument. In other words, each piston assembly can move independently of other piston assemblies. The degree of freedom refers to the number of independent movements that the medical instrument enables. Preferably, the piston assemblies are arranged side-by-side. In particular, the piston assemblies may be positioned in a space-saving manner so that they take up only a small amount of installation space.

In some embodiments, the drive-side coupling device may comprise at least one drive unit which is adapted to drive and/or move the at least one piston assembly or lever device. The drive unit may be adapted to transmit at least one drive force and/or at least one drive torque to the piston assembly and/or the lever device. Preferably, each lever device may be coupled to a respective drive unit. The drive unit may comprise at least one drive shaft, at least one gear transmission, at least one belt drive, at least one cable drive, at least one chain drive, and/or at least one rack-and-pinion transmission. The drive unit may further comprise at least one electromagnet, at least one piezo actuator, at least one hydraulic actuator, and/or at least one pneumatic actuator. In an alternative embodiment, each piston or piston assembly may be coupled to a respective drive unit. In the coupled state, the drive unit may be controllable from a robot side.

According to a further development, the at least one instrument-side coupling device may comprise at least one piston assembly which is complementary to the at least one drive-side piston assembly and is adapted to absorb movements of the at least one drive-side piston assembly and/or to transmit movements to the at least one drive-side piston assembly. The at least one complementary piston assembly may be arranged in an instrument-side housing. The complementary piston assembly may comprise at least one complementary piston and at least one further complementary piston.

Apart from the interposed medical barrier, the transmission of the movements can take place directly from a drive-side piston to an instrument-side piston or vice versa, and/or indirectly—for example, by the pistons actuating, in particular, hydraulic and/or pneumatic systems that interact with each other.

In order to prevent or at least minimize damage and/or wear within the interface, the at least one complementary piston assembly may have at least one edge which is rounded at least in sections, preferably completely. In particular, by means of such a design, damage to the medical barrier and potentially associated contamination can be advantageously avoided.

According to a further development, the piston device may comprise at least one elastic membrane which is adapted to cover the end face of a piston at least in sections. Such a design reduces the risk of damage to the medical barrier, because it prevents the medical barrier from becoming trapped between the piston and its cylinder guide. Due to the elasticity of the membrane, it can be mechanically, hydraulically, and/or pneumatically deformed or bulged and/or compressed, and thus transmit and/or absorb movements or mechanical energy.

The complementary piston assembly may further comprise at least one instrument-side spring device which is designed to axially preload the at least one complementary piston and/or the at least one further complementary piston in a spring-loaded position, to ensure a reliable coupling with the drive-side piston assembly. In some embodiments, both the piston assembly and the complementary piston assembly may comprise at least one spring device.

A further aspect of the disclosure relates to a medical robot system comprising an interface of the type described above.

By means of such an interface, an advantageously further developed medical system can be provided. In particular, installation and/or deinstallation can be simplified and costs and downtimes can be reduced, because a continuous medical barrier is used that is, in particular, not complexly formed and/or has no specially embedded adapters for transmitting a driving force and/or a driving torque. This makes it possible to eliminate the need for time-consuming, precise placement of the medical barrier. In addition, the cost of each medical application can be reduced, because a single-use medical barrier can be kept as cost-effective as possible. In particular, a standard medical barrier can be used. Furthermore, the features according to the disclosure can provide a particularly reliable and functional interface with optimized properties, so that a sterile working field is always guaranteed or ensured, in particular before and/or during a medical procedure on a patient.

The devices and systems according to the disclosure and the methods according to the disclosure are hereby not to be limited to the application and embodiment described above. In particular, they can have a number of individual elements, components, and units which differ from a number mentioned herein, in order to fulfill a function described herein. In addition, for the ranges of values specified in this disclosure, values within the stated limits shall also be deemed to be disclosed and to be usable in any manner.

The present disclosure is described below by way of example with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using numerical words, such as first, second, third object, etc., these are used to name and/or assign objects. Accordingly, for example, a first object and a third object may be included, but not a second object. However, a number and/or sequence of objects could also be derived using numerical words.

### BRIEF DESCRIPTION OF DRAWINGS

FIG. 1 shows a medical robot system with an interface for connecting a drive unit to a medical instrument,

FIG. 2 is a perspectival view of the interface with a drive-side coupling device and an instrument-side coupling device in a state in which they are placed against each other,

FIG. 3 is a side view of the drive-side coupling device and the instrument-side coupling device in a spatially separated state,

FIG. 4 is a perspectival view of the interface with the medical barrier inserted,

FIG. 5 is a perspectival view of part of the drive-side coupling device with the housing removed,

FIG. 6 is a detail view of an embodiment of a piston assembly of the drive-side coupling device,

FIG. 7 is a schematic, longitudinal sectional view of a part of the drive-side coupling device,

FIG. 8 is a perspectival view of the instrument-side coupling device with focus on an instrument-side coupling surface,

FIG. 9 is a perspectival view of the drive-side coupling device with focus on a drive-side coupling surface,

FIG. 10 is a perspectival view of the instrument-side coupling device with the drive-side piston assembly coupled thereto,

FIG. 11 is a perspectival view of the interface in the coupled state,

FIG. 12 a detail view of the interface in the coupled state,

FIG. 13 is a schematic view of another embodiment of an interface in the coupled state,

FIG. 14 is a sectional view through another embodiment of a piston assembly,

FIG. 15 is a schematic, longitudinal sectional view according to another embodiment,

FIG. 16 is a schematic, longitudinal sectional view of the interface according to FIG. 15 in the coupled state,

FIG. 17 is a flowchart of a method for connecting the coupling devices, with the medical barrier interposed.

### DETAILED DESCRIPTION OF PREFERRED EMBODIMENTS

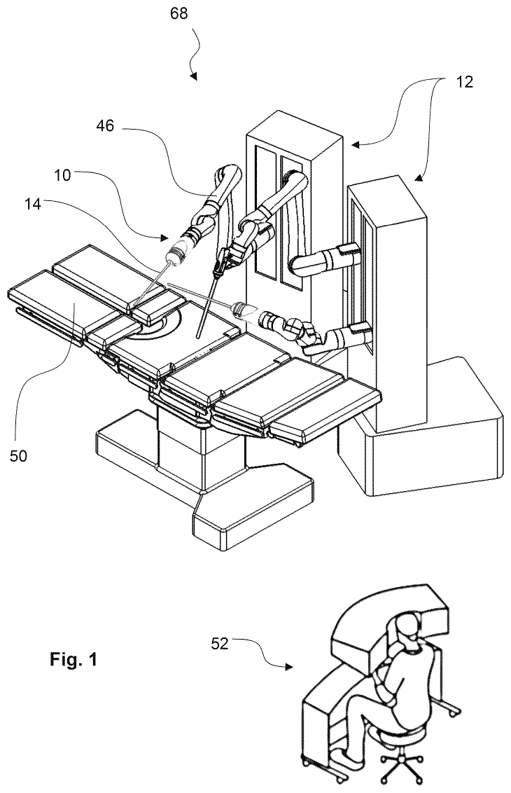

FIG. 1 shows a medical robot system 68. The robot system 68 has a patient bed 50. The robot system has a robot arm 46 and a drive unit 12, wherein the robot arm 46 can be controlled via the drive unit 12. The drive unit 12 can be actuated automatically, semi-automatically, and/or by medical personnel by means of an operating console 52.

The robot system has a medical instrument 14 which is arranged on the robot arm 46 and can be moved thereby. The medical instrument 14 itself has at least one movable part which is movable relative to another part of the medical instrument 14. The movable part is movable relative to the other part by the drive unit 12. In the present case, the medical instrument 14 is a pair of forceps, although any other instruments deemed appropriate by a person skilled in the art would also be conceivable. For the sake of clarity, not all components of the robot system are provided with reference signs.

In order to create sterile working conditions, a continuous medical barrier 24 is provided which separates a region around the patient bed 50 from the potentially non-sterile drive unit 12 and the robot arm 46. A driving force and/or a driving torque for the movable part of the medical instrument 14 is transmitted by means of an interface 10 of the robot system. In a use state, the interface 10 is arranged in the region of the medical barrier 24. In the use state, the interface 10 is held and guided by the robot arm 46.

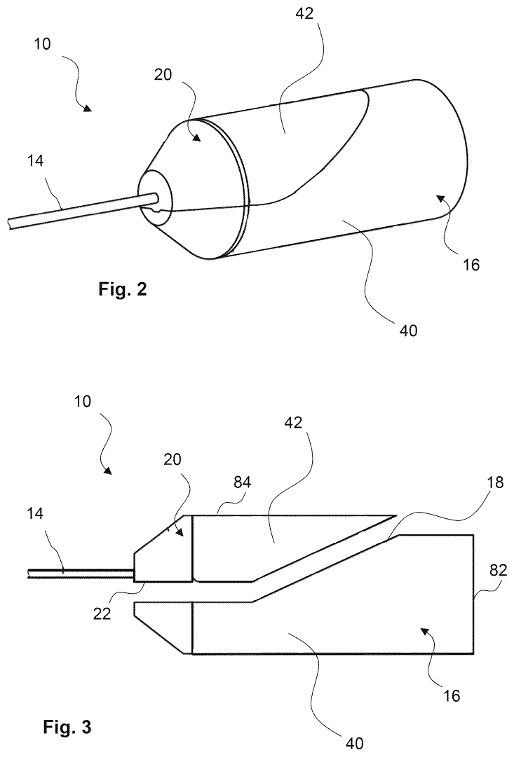

The interface 10 has a drive-side coupling device 16 and an instrument-side coupling device 20, which are shown in FIG. 2 in a perspectival view in a state in which they are placed against each other. FIG. 3 shows the coupling devices 16, 20 in a spatially separated state. Furthermore, the coupling devices 16, 20 each have a housing 82, 84, which can be connected to one another in a captive manner. The connection between the two housings 82, 84 is releasable.

The drive-side coupling device 16 has a drive-side coupling surface 18. The instrument-side coupling device 20 comprises an instrument-side coupling surface 22. The coupling surfaces 18, 22 are complementary to each other and allow a positive connection of the connecting elements 16, 20, as shown in FIG. 2.

The coupling devices 16, 20 or their housings 82 and 80, when assembled, form an at least partially circular-cylindrical and at least partially frustoconical body (cf. FIGS. 2 and 3).

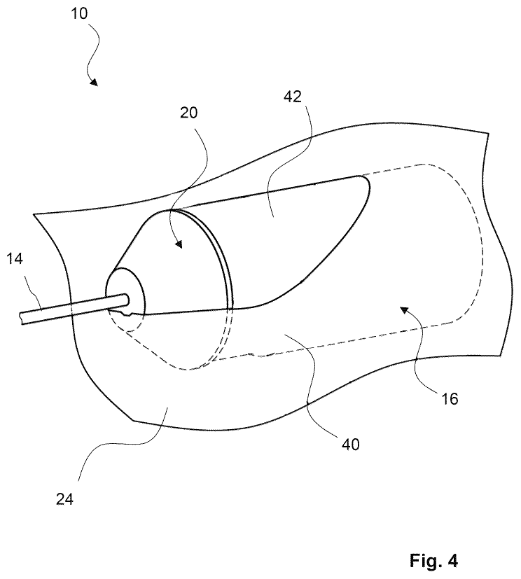

The coupling surfaces 18, 22 are adapted to clamp the medical barrier 24 (cf. FIG. 4). The coupling devices 16, 20 can be coupled to one another via their respective coupling surfaces 18, 22, with the interposition of the medical barrier 24. The medical barrier 24 is a continuous plastic film which is in particular free of openings. In the use state, the two coupling devices 16, 20 are separated from each other by the medical barrier 24. In the use state, the instrument-side coupling device 20 together with the medical instrument 14 is located on a sterile side of the medical barrier 24. In the use state shown in FIG. 4, the drive-side coupling device 16 is largely concealed by the medical barrier 24, which is indicated by the dashed lines.

The instrument-side coupling device and/or its housing can be held together in a captive manner with the drive-side coupling device and/or its housing, for example, by means of a clamp (not shown here) and/or by means of another advantageous, preferably reversible, fastening device.

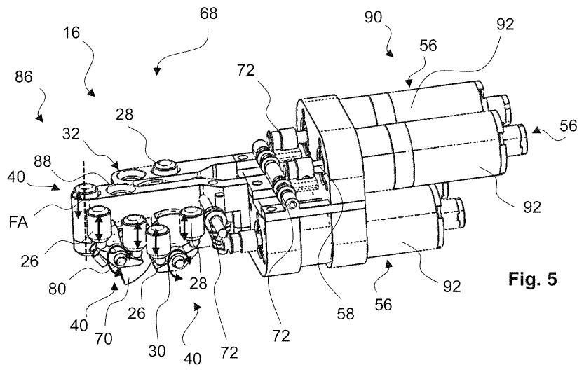

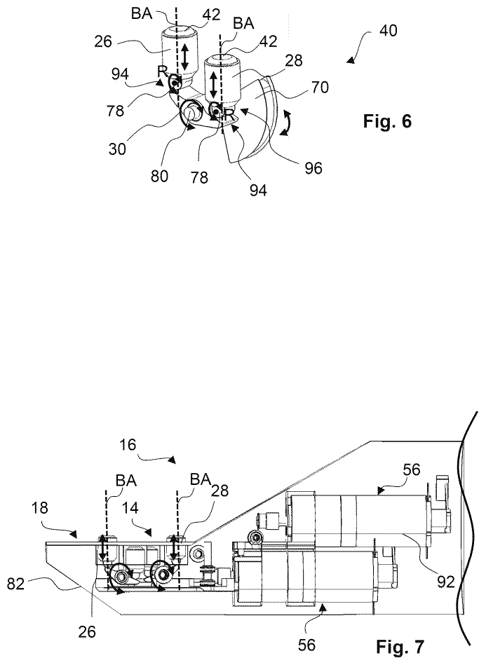

According to the disclosure, a transmission of a driving force and/or a driving torque through the continuous medical barrier 24 occurs mechanically via a piston assembly 40. In FIG. 5, a perspectival view of a part of the drive-side coupling device 16 with a total of four drive-side piston assemblies 40 in a section 86 of the coupling device can be seen. For illustration purposes, the housing 82 of the drive-side coupling device 16 has been hidden. FIG. 5 therefore shows the interior of the drive-side coupling device 16. Each of the four drive-side piston assemblies 40 comprises two pistons 26, 28, which are interdependently coupled to one another via a lever device 30 (cf. FIG. 6). The piston assemblies 40 are positioned such that all pistons 26, 28 have the same spatial orientation, but are offset parallel to one another. Furthermore, a cylinder guide 32 can be seen. In order to better identify the individual components, a front part of the cylinder guide 32 has been hidden. The cylinder guide 32 has boreholes 88 in this case. Each of the boreholes 88 is designed to guide a piston 26, 28 along a substantially vertical guide axis FA and to prevent movement in the horizontal direction. The degrees of freedom are indicated by arrows.

In a section 90, the drive-side coupling device 16 shown in FIG. 5 has a total of four drive sources 56, wherein the fourth is arranged hidden behind the three drive sources 56 that can be seen. The drive sources 56 are designed as direct current motors 92. It goes without saying that, in other embodiments, depending upon the area of application, other drive sources can be used alternatively or additionally. In particular, the at least one drive source 56 may additionally or alternatively comprise at least one electromagnet, at least one piezo actuator, at least one hydraulic actuator, and/or at least one pneumatic actuator. The drive sources 56 are arranged parallel to one another and extend substantially in a horizontal spatial direction towards the drive-side piston assemblies 40.

In the embodiment shown here by way of example, the drive-side coupling device 16 further comprises belt drives which are each designed to drive a drive-side piston assembly 40 and/or a lever device 30. Each drive source 56 is coupled to a respective drive-side piston assembly 40 in a torque-transmitting manner via a respective belt drive. Each belt drive includes a drive source 56 with a drive shaft 58. Furthermore, each belt drive comprises a drive device in the form of a driven pulley 70, which is arranged on an output shaft 80. The output shaft 80 is coupled to the lever device 30 in a torque-transmitting manner. In order to transmit a torque or a force from the drive source 56 to the drive-side piston assembly 40, the drive-side coupling device 16 comprises at least one belt (not shown here) which connects one of the drive shafts 58 to a corresponding driven pulley 70. In order to hold the belt in the correct position and to guide its movement, to ensure efficient power transmission and to use the installation space efficiently and in a space-saving manner, the drive-side coupling device 16 also has deflection rollers 72. Alternatively or additionally, other gear types may also be provided. In particular, worm gears, Geneva gears, hydraulic gears, pneumatic gears, and/or gear drives can also be used. For the sake of clarity, not all components of the drive-side coupling device 16 are provided with reference signs. FIG. 6 shows a detail view of an embodiment of a drive-side piston assembly 40 according to FIG. 5. The piston assembly 40 includes the output axle 80, along which the lever device 30 and the output pulley 70 are arranged. The lever device 30 has two lever ends 94 or projections which extend radially away from the output axle 80 in opposite spatial directions. On each of the two lever ends 94, one of the pistons 26, 28 is arranged, the end faces 42 of which face away from the lever device 30. On their undersides 96, the pistons 26, 28 have cams 78 via which they contact the lever ends 94. The cams 78 are rotatably connected to the lever device 30, so that the pistons 26, 28 always maintain the same orientation or alignment in the event of a rotational movement of the lever device 30 about a rotation axis R which extends into the plane of the drawing. Via the lever device 30, the two pistons 26 are interdependent. In other words, the two pistons 26, 28 cannot be moved independently of each other. However, due to the design of the lever device 30, the directions of movement of the pistons 26, 28 are always contrary or opposite.

FIG. 7 shows a schematic, longitudinal sectional view of the drive-side coupling device 16. The compact and space-saving arrangement of the drive-side piston assemblies 40, drive sources 56, and gears in a housing 82 can be seen. It can also be seen that the reference axes BA along which the pistons are movable run perpendicular to the drive-side coupling surface 18. In the embodiment shown, the pistons 26, 28 are adapted to move out of the drive-side coupling surface 18 at least in sections.

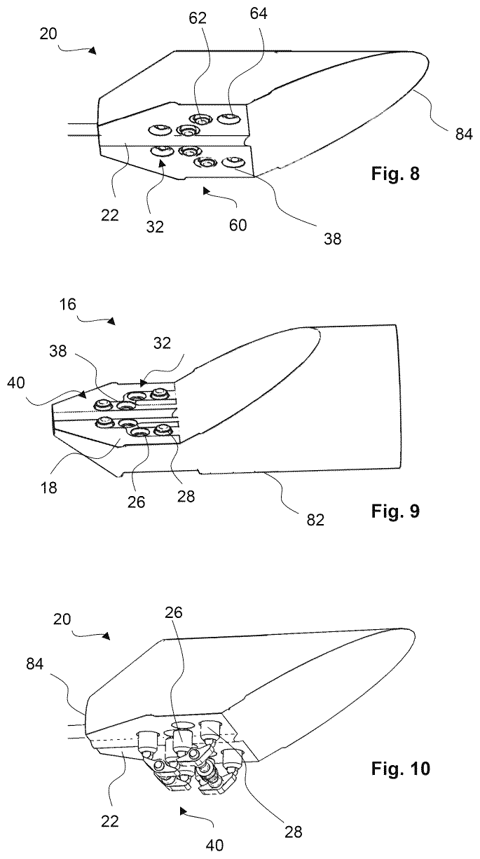

FIGS. 8 and 9 show a perspectival view of the instrument-side coupling device 20 or drive-side coupling device 16 with a focus on the respective coupling surfaces 18, 22. It can be seen that the instrument-side coupling device 20 comprises at least one instrument-side piston assembly 60 which is complementary to the at least one drive-side piston assembly 40 and is adapted to absorb movements of the at least one drive-side piston assembly 40 and/or to transmit movements to the at least one drive-side piston assembly 40. The complementary instrument-side piston assembly 60 comprises at least one complementary instrument-side piston and at least one further complementary instrument-side piston 62, 64.

As will be explained further below, the complementary pistons 62, 64 do not necessarily have to be coupled to one another via a lever device 30. The instrument-side coupling device 20 also has cylinder guides 32 which are designed to be complementary to the drive-side cylinder guides 32. The cylinder guides 32 of the instrument-side coupling device 20 and the drive-side coupling device 16 are, in particular, designed or arranged such that, in a state of the interface 10 in which the drive-side coupling device 16 is coupled to the instrument-side coupling device 20, they are congruent, so that a piston 26, 28 of the drive-side coupling device can penetrate at least partially into the instrument-side complementary cylinder guide 32 in the coupled state of the interface 10. Advantageously, all edges 38 of the coupling device 16, 20 and in particular all edges 38 of the coupling surfaces 18, 22 and/or the piston assemblies are rounded. This can reduce the risk of damage and the associated contamination of the work area.

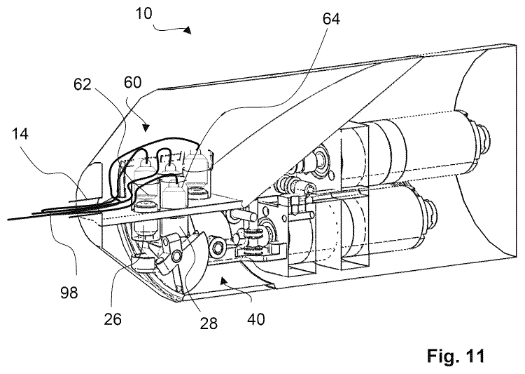

A functional coupling of the drive-side piston assemblies 40 with the complementary instrument-side piston assembly 60 is shown as an example in FIG. 10. For the sake of clarity, the drive source 56, the gear assembly, and the housing 82 of the drive-side coupling device 16 have been hidden in this illustration. The medical barrier 24 is also not shown.

FIG. 11 shows a perspectival view of an interface 10 in the coupled state. The drive-side pistons 26, 28 are in physical contact with the complementary instrument-side pistons 62, 64. In the embodiment shown, the complementary instrument-side pistons 62, 64 are further coupled to pull cables 98 to impart movements to a medical instrument 14 connected to the pull cables 98. By pulling or pushing the pull cables 98, the position of a medical instrument 14 and/or its actuators can be controlled precisely and/or remotely. In further embodiments not shown here, the interface 10 may be provided with additional or alternative coupling mechanisms, which are advantageous for a specific application, between the complementary instrument-side pistons 62, 64 and the medical instrument 14 or its effectors. The medical barrier 24 is also not shown in this illustration.

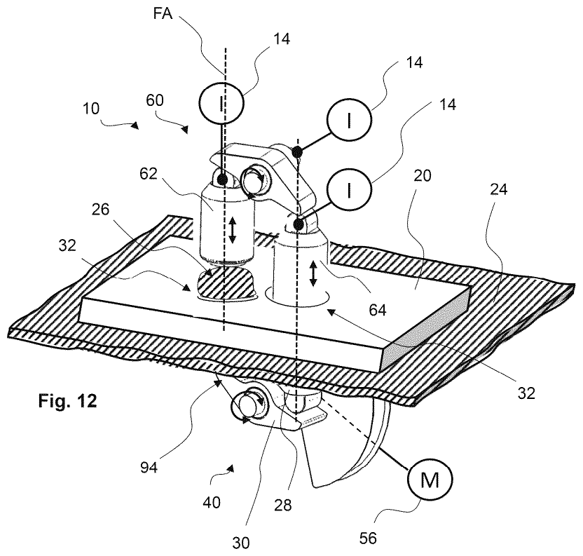

FIG. 12 shows a detail view of the interface 10 in the coupled state. The drive-side piston assembly 40 can be seen, which is mechanically coupled to a complementary instrument-side piston assembly 60, with the interposition of a medical barrier 24. By actuating the lever device 30, the left drive-side piston 26 is pushed upwards by the left lever end 94. The piston 26 is moved through a cylinder guide 32 along a guide axis FA. Furthermore, the piston 26 presses into a section of the medical barrier 24, which covers the cylinder guide 32, in the coupled state of the interface 10, in the instrument-side coupling device 20 or its complementary cylinder guide 32. Since the drive-side pistons 26, 28 are mechanically coupled to the instrument-side pistons 62, 64, the raised left drive-side piston 26 or, in the present case, the interposed medical barrier 24 exerts a force on the instrument-side, complementary left piston 60. Since the resulting force from the drive-side lever device 30 acts upon both the drive-side piston 26 and the complementary instrument-side piston 60, both pistons 26, 60 are moved in the same direction. The right instrument-side piston 62 is interdependently coupled to the left instrument-side piston 64 via a lever device 30, such that the latter is pressed downwards. The right piston 62 presses the medical barrier 24 into a drive-side cylinder guide 32, which is hidden by the medical barrier 24 in the illustration. The right instrument-side piston 62 presses on the right drive-side piston 28, such that the latter is also pressed downwards. It goes without saying that the direction in which the lever device 30 can be actuated can vary. In the embodiment shown here, the mechanical force and/or movement transmitted to the instrument-side pistons 62, 64 can then be transmitted to the end effector of the medical instrument 14 via a gear not shown in this illustration, such that the end effector executes a corresponding movement. In alternative embodiments, the instrument-side pistons 62, 64 may be coupled directly to the end effector of the medical instrument 14 via, for example, pull cables 98, and in this way transmit mechanical energy.

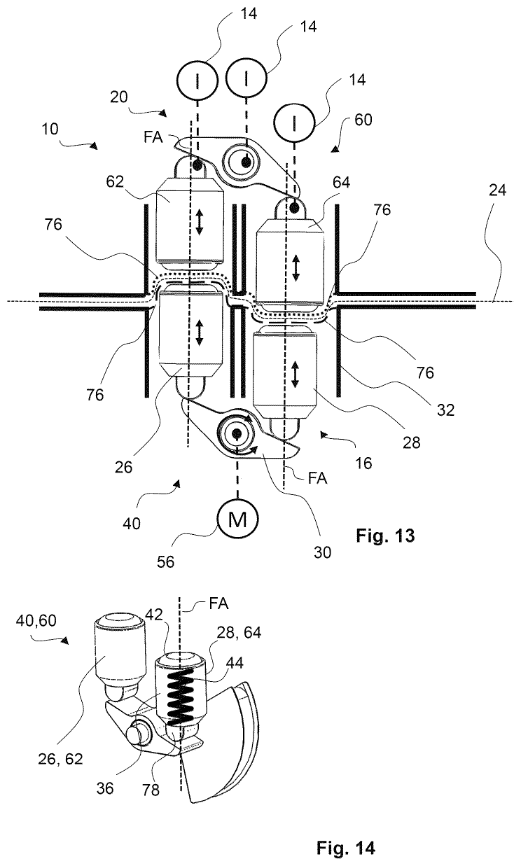

FIG. 13 shows a schematic representation of another embodiment of an interface 10 in the coupled state. According to the embodiment shown therein, the interface 10 comprises a drive-side coupling device 16 and a complementary instrument-side coupling device 20 with elastic membranes 76. The elastic membranes 76 are adapted to cover at least portions of end faces 42 of a piston 26, 28, 62, 64. Such a design reduces the risk of damage to a medical barrier 20 because it prevents the medical barrier 20 from becoming trapped and possibly damaged—for example, between the piston 26, 28, 62, 64 and its cylinder guide 32. Due to the elasticity of the membranes 76, they can be mechanically bulged and/or compressed and thus transmit and/or absorb movements or mechanical energy. In alternative or additional embodiments not shown here, the membranes can be deformed and/or bulged—for example, hydraulically and/or pneumatically.

FIG. 14 shows a detail view of a further embodiment of a piston assembly 40, 60. The embodiment shown here can be used both on the drive side and on the instrument side. The embodiment of the piston assembly 40, 60 shown here comprises a spring device 44 which is designed to axially preload at least one complementary instrument-side piston 62 and/or at least one further complementary instrument-side piston 64 or at least one drive-side piston 26 and/or at least one further drive-side piston 28 in a spring-loaded position, to always ensure a reliable coupling with the drive-side piston assembly 40. In the embodiment shown herein, the spring device 44 is arranged between a cam 78, which contacts a lever device, and an end face 42 of the piston 28, 64. The end face 42 of the piston 26, 62 is connected to the spring device 44. Furthermore, the end face 42 is movable relative to a piston wall 36 along a guide axis FA. At this point it should be noted that the embodiment shown is merely an example. The spring device 44 does not necessarily have to be integrated into the piston 26, 28, 62, 64, as shown in FIG. 13; alternatively or additionally, it can also advantageously be arranged outside the piston 26, 28, 62, 64—for example, between a cam 78 and an underside 96 of the piston 26, 28, 62, 64. In some embodiments, the piston 26, 28, 62, 64 itself may be designed, at least in large part, as a spring device 44.

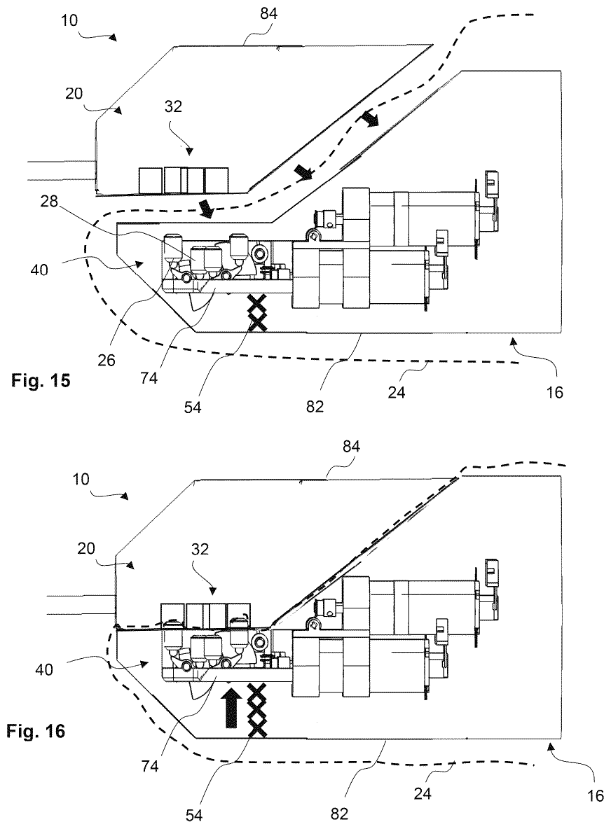

A further development of the interfaces 10 described above is shown in FIGS. 15 and 16. FIGS. 15 and 16 show a schematic longitudinal sectional view of an interface 10 in which a drive-side coupling device 16 comprises at least one lifting device 54 which is adapted to lift at least one drive-side piston assembly 40 and/or at least one drive-side coupling device 16, in order to operatively couple the drive-side coupling device 16 to an instrument-side coupling device 20. Furthermore, such a lifting device 54 facilitates the proper coupling process. In the embodiment shown, the lifting device 54 is arranged below the drive-side piston assembly 40. The lifting device 54 is firmly connected to a housing 82 of the drive-side coupling device 16 and to a rod 74, on which the drive-side piston assembly 40 or a gear is positioned or attached. It goes without saying that the position of the lifting device 54 is by no means limited to the embodiment shown here. Furthermore, the drive-side piston assembly 40 is positioned in a non-coupled state in the housing 82 of the drive-side coupling device 16 such that no piston 26, 28 of the coupling device 16 protrudes from a drive-side coupling surface 18. In this rest or transport state, the sensitive mechanics within the housing 82 of the drive-side coupling device 16 are largely protected from external influences.

An interface 10 according to this embodiment may be adapted to detect whether the drive-side coupling surface 18 has been properly connected to an instrument-side coupling surface 22. If this is the case, the lifting device 54 can be activated and raise the drive-side piston assembly 40 into an operating position. By means of the lifting process, at least a portion of the at least one and/or the at least one further drive-side piston 26, 28 can be pressed into the instrument-side coupling device 20, in particular into a complementary instrument-side cylinder guide 32. In addition or as an alternative to the embodiment described herein, the instrument-side coupling device 20 may also comprise a lifting device 54 which is correspondingly adapted to press the complementary instrument-side piston assembly 60 into the drive-side coupling device 16, in particular into the drive-side cylinder guide 32.

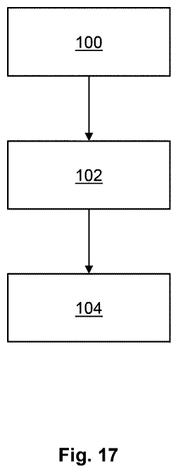

FIG. 17 shows a flowchart of a method for connecting the coupling devices 16, 20, with the medical barrier 24 interposed. In a step 100, the first coupling surface 18 and the outer surface 82 of the drive-side connecting element 16 are at least partially covered with the medical barrier 24. In a step 102, the instrument-side connecting element 20, via its second coupling surface 22, with the medical barrier 24 in-between, is connected to the first coupling surface 18. Finally, in a step 104, the lifting device 54 is actuated, and the interface 10 is transferred to a use state.

1. An interface for connecting a drive unit to a medical instrument, comprising:
at least one drive-side coupling device with at least one first drive-side coupling surface, and
at least one instrument-side coupling device, complementary to the drive-side coupling device, with at least one instrument-side coupling surface,
wherein the drive-side and the instrument-side coupling devices can be mechanically coupled to one another with the interposition of a continuous medical barrier, and
wherein at least the drive-side coupling device includes at least one drive-side piston assembly with at least one piston which is adapted to be moved alternately in opposite directions.

2. The interface according to claim 1, wherein the alternating movement takes place along a reference axis which runs obliquely or perpendicular to the drive-side coupling surface.

3. The interface according to claim 1, wherein the drive-side piston assembly includes at least one further piston which is interdependently coupled to the at least one piston.

4. The interface according to claim 2, further including: at least one lever device, wherein the at least one piston can be actuated together with the at least one further piston via the at least one lever device.

5. The interface according to claim 1, wherein the at least one drive-side piston assembly includes at least one cylinder guide which is adapted to guide the at least one piston and/or the at least one further piston during a movement along a guide axis.

6. The interface according to claim 5, wherein each cylinder guide includes at least one guide sleeve.

7. The interface according to claim 5, wherein the at least one cylinder guide has at least one edge which is rounded at least in sections, preferably completely.

8. The interface according to claim 1, wherein the at least one piston and/or the at least one further piston has at least one end face whose edge is rounded at least in sections, preferably completely.

9. The interface according to claim 1, wherein the drive-side piston assembly includes at least one spring device which is adapted to axially preload the at least one piston and/or the at least one further piston in a spring-loaded position to ensure reliable movement transmission to the medical instrument.

10. The interface according to claim 1, comprising at least one lifting device which is adapted to raise at least the at least one drive-side piston assembly and/or the drive-side coupling device to operatively couple the drive-side coupling device to the instrument-side coupling device.

11. The interface according to claim 1, wherein the drive-side coupling device includes a plurality of drive-side piston assemblies.

12. The interface according to claim 11, wherein each drive-side piston assembly is assigned a degree of freedom of the medical instrument.

13. The interface at least according to claim 4, wherein the drive-side coupling device includes at least one drive source which is adapted to drive the lever device.

14. The interface according to claim 13, wherein the drive source includes at least one drive shaft, at least one gear transmission, at least one belt drive, at least one cable drive, at least one chain drive, and/or at least one rack-and-pinion transmission.

15. The interface according to claim 13, wherein the drive source includes at least one electromagnet, at least one piezo actuator, at least one hydraulic actuator, and/or at least one pneumatic actuator.

16. The interface according to claim 1, wherein the at least one instrument-side coupling device includes at least one instrument-side piston assembly which is complementary to the at least one drive-side piston assembly and is adapted to absorb movements of the at least one drive-side piston assembly and/or to transmit movements to the at least one drive-side piston assembly.

17. The interface according to claim 16, wherein the complementary instrument-side piston assembly includes at least one complementary instrument-side piston and at least one further complementary instrument-side piston.

18. The interface according to claim 16, wherein the at least one complementary instrument-side piston assembly has at least one edge which is rounded at least in sections, preferably completely.

19. The interface according to claim 17, wherein the complementary instrument-side piston assembly includes at least one instrument-side spring device, which is adapted to axially preload the at least one complementary instrument-side piston and/or the at least one further complementary instrument-side piston in a spring-loaded position to ensure a reliable coupling with the drive-side piston assembly.

20. A medical robot system comprising an interface according to claim 1.
